(19) United States
(12) Patent Application Publication
(10) Pub. No.: US 2010/0168823 A1
(43) Pub. Date: Jul. 1, 2010

(54) METHOD AND APPARATUS FOR THE TREATMENT OF RESPIRATORY AND OTHER INFECTIONS USING ULTRAVIOLET GERMICIDAL IRRADIATION

(76) Inventor: John Strisower, Chico, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/648,113

(22) Filed: Dec. 28, 2009

Related U.S. Application Data
(63) Continuation of application No. 11/053,526, filed on Feb. 7, 2005.
(60) Provisional application No. 60/543,588, filed on Feb. 9, 2004.
(60) Provisional application No. 60/550,631, filed on Mar. 4, 2004.
(60) Provisional application No. 60/553,040, filed on Mar. 12, 2004.

(51) Int. Cl. A61N 5/067

(52) U.S. Cl. 607/89

(57) ABSTRACT

Method and apparatus for using computer controlled, fiber-coupled laser delivery of treatment specific wavelength, intensity and duration of UV irradiation to control bacterial, fungal, viral and mold infections in bodily cavities, fluids and external applications. The method of treatment is focused on DNA breakdown beyond repair by natural DNA repair mechanisms of the pathogen, with less than damaging doses to tissues being treated, thus avoiding mutagenicity and carcinogenicity. The minimal intensity and duration and exposure area of any given surface of tissue to be treated is to be pre-determined by tissue and pathogen testing to optimize the therapeutic ratio. External applications include specifically Trichophyton Rubrum (toenail fungus) through the nail and Pseudomonas Aeruginosa infections in burns and elsewhere.

### RELATED APPLICATIONS

This application is a continuation of application Ser. No. 11/053,526, filed on Feb. 7, 2005, which is incorporated herein by reference for all purposes and which claims priority to Provisional Application Ser. No. 60/543,588 filed on Feb. 9, 2004, Provisional Application Ser. No. 60/550,631 filed on Mar. 4, 2004 and Provisional Application Ser. No. 60/553,040 filed on Mar. 12, 2004, which are incorporated herein by reference for all purposes.

### BACKGROUND

The disclosed method and apparatus relates generally to methods and apparatus for the treatment of respiratory, blood or other body cavity infections in humans and/or animals, and/or inanimate object disinfection. It has been known for almost 100 years that ultraviolet light in the 248-253.7 nm wavelength range, the so called deep or far ultraviolet (also known as UVC), is lethal in small doses of short time duration, meaning power level per area exposed over time, to most bacteria, viruses, fungi and molds. An approximate band that is useful in the applications of the disclosure of this patent is the band from about 200 nm to 320 nm. DNA deactivation appears to be somewhat more likely or more efficient in the shorter wavelength part of this range, from about 200 nm to 250 nm. Antibiotics delivered orally or by intravenous methods are somewhat effective at eradicating certain pathogens in the lung tissue where the circulatory system is able to deliver the drug. However, the larger airways of the lungs (and certain other body or organ cavities) are not particularly accessible via the circulatory system. Further, the larger airways of the respiratory system (trachea and major bronchi) are the predominant producers of mucous which create a protein rich environment for pathogen growth that is physically distant from vascular access.

The overall disclosure herein is using computer controlled, fiber-coupled laser delivery of treatment specific wavelength, intensity and duration of UV irradiation to control bacterial, fungal, viral, and mold infections in bodily cavities, fluids and external applications. The method of treatment is focused on DNA breakdown beyond repair by natural DNA repair mechanisms of the pathogen, with less than damaging doses to tissues being treated, thus avoiding mutagenicity and carcinogenicity. The minimal intensity and duration and exposure area of any given surface of tissue to be treated is to be pre-determined by tissue and pathogen testing to optimize the therapeutic ratio. External applications include specifically Trichophyton Rubrum (toenail fungus) through the nail and Pseudomonas Aeruginosa infections in burns and elsewhere.

The disclosure herein is, additionally, for a surgically installed inline arterial blood treatment device that allows for outpatient and in-home application of computer controlled, preprogrammed therapies of UV germicidal irradiation via a fiber optic connection external to the patient's body. With a simple fiber optic connector, the computer controlled, fiber optic coupled laser UV light source delivers the desired wavelength, intensity and duration needed to deactivate pathogens (bacterial, viral and others) in blood as it traverses through the device. The method of treatment is focused on DNA breakdown beyond repair by natural DNA repair mechanisms of the pathogen, with less than damaging doses to tissues being treated, thus avoiding mutagenicity and carcinogenicity. Further, as blood cells do not reproduce but rather are generated in bone marrow, their need for DNA to reproduce is unimportant while the pathogens attached to the blood cells are then unable to replicate thereby reducing further colonization of new blood cells.

Further still, the disclosure herein is for using perflourocarbons and other possible partial liquid ventilation substances, doped with optically appropriate compounds to reflect and refract UV light delivered via Ultraviolet Video Bronchoscopic Devices to allow UV germicidal irradiation of remote and difficult to reach spaces within the respiratory system. The method of treatment is focused on DNA breakdown beyond repair by natural DNA repair mechanisms of the pathogen, with less than damaging doses to tissues being treated, thus avoiding mutagenicity and carcinogenicity. Additionally, these perflourocarbons and other possible partial liquid ventilation substances can be used as a means of transport of retrovirus vectors to deliver gene therapies to difficult to reach areas within the respiratory system thereby enabling an effective therapeutic outcome previously not possible.

When used in a lung treatment application, the disclosure incorporates a fiber optic coupled, computer controlled light source or laser emitting UVC via a video bronchoscope or other suitable device for insertion into a patient's lungs. The computer controller is capable of determining the frequency or wavelength of light and the power of the light applied as indicated by the patient's condition and size, tissue being treated, amount of mucous present and pathogen type. Almost all viruses, bacteria and fungi are killed by 253.7 nm wavelength of UVC but other wavelengths are probably even more beneficial and efficient. The disclosure provides for methods for the pulmonologist or other medical professional to apply the treatment in a systematic manner such that all areas of potential pathogen colonization are exposed to the predetermined duration, intensity and wavelength of UVC light. The method also specifies that the pulmonologist or other appropriate medical professional, using a video bronchoscope monitor, can control the instrument placement into the distal end of each of the third generation major bronchial branches. The computer controller can then be set to deliver the desired wavelength, duration and intensity of UVC as the instrument is withdrawn smoothly and slowly enough to evenly expose the infected airway region. Withdrawal can be by hand or by suitable mechanical or electromechanical devices. For example, an electromechanical withdrawal device can be devised using an exposure power level versus time function built into the monitor or other hardware of the apparatus so the practitioner can be more certain that the withdrawal was at the right or optimal speed. Once the instrument is withdrawn to the proximal end of the branch where it meets the next higher generation bronchial branch, the light source is turned off. In practice, one way to implement this is to provide the light source with a shutter on the fiber coupling and/or the PC controller which would be able to control the light without powering off the light source. Next, the instrument is inserted into the next higher third generation bronchial branch to the distal extent accessible and this process is repeated for all 18 of the segmental bronchi airways, followed by similar treatment of the right and left main bronchi and finally the trachea as the procedure is completed.

### BRIEF DESCRIPTION OF THE DRAWINGS

FIG. 1 illustrates a video bronchoscope capable of reaching the distal end of all 18 of the segmental bronchi in pediatric or adult patients.

FIG. 2 illustrates the disclosed apparatus shown as a modification to or accessory for a video bronchoscope where either one of the fiber optic light sources normally used to provide light for video bronchoscopy is setup for UVC delivery (in combination with or instead of visible light while UVC is being delivered) or the accessory channel is used for the fiber optic delivery of the UVC light to the desired location. Additionally, the light source and computer controller are depicted.

FIG. 3 illustrates the major airways of the human respiratory system 300 that are of primary interest to the disclosure of this patent for lung disease applications.

FIG. 4 is a more detailed illustration of the major airways of the human respiratory system, specifically illustrating the peripheral bronchi.

FIG. 5 illustrates red blood cells treatable by one embodiment of the disclosure.

FIG. 6 illustrates a device for blood treatment using an embodiment of the patent.

FIG. 7 illustrates a second device for blood treatment using the teachings of the patent.

FIG. 8 illustrates an additional embodiment for bodily fluid treatment.

### SUMMARY OF THE INVENTION

The disclosed method and apparatus provides useful methods and apparatus for the treatment of respiratory, or other, pathogen infections using ultraviolet light germicidal irradiation (UVGI) as a germicidal agent and can be used in combination with traditional antibiotic and other drug therapies. The smaller airways and lung tissues are better suited to infection treatment using antibiotics due to their inherent vascular accessibility. The combination of drugs and UVGI of the larger airways provides more complete pathogen eradication with greatly reduced risk of re-infection or at least longer durations of reduced symptoms while pathogen colonies regenerate between treatments. In addition to respiratory therapy, the disclosed method and apparatus can also be used in the treatment of blood infections, and other body cavity infections in humans and/or animals, and/or inanimate object disinfection.

### DETAILED DESCRIPTION

The disclosure generally pertains to methods and apparatus for the reduction and/or elimination of pathogens causing infection in human and animal respiratory systems and other body cavities. The disclosure is applicable to the disinfection of difficult to reach and access areas of inanimate objects as well. Further, the disclosed method and apparatus is applicable to heart-lung and blood transfusion systems for pathogen and/or chemical antigen deactivation in blood by exposing the blood cells to UVC at such a wavelength and intensity and duration as to deactivate the antigen. This can be accomplished via a UVC venous system wherein multiple simultaneous UVC tubes are used to exposure a large volume of blood simultaneously. The disclosure utilizes apparatus comprised of a computer controllable UVGI light-source fiber optically delivering the light to desired areas via an accessory for or modification to existing video bronchoscopes. The computer can control the duration, intensity and wavelength(s) of light being delivered during treatments. The disclosure includes methods for treatments of infected areas in a systematic manner that assures maximum pathogen kill ratios with minimal risk of tissue damage. The disclosed method and apparatus is designed to work in conjunction with antibiotic drug therapies wherein the drugs perform the primary function of disinfecting small airways and tissue that are vascular and accessible via the circulatory system. The disclosure provides the methods and apparatus to disinfect larger airways where greater mucous quantities are produced that creates an opportune environment for pathogen colonization and where the circulatory system does a poor job of delivery of intravenous or orally administered antibiotics. By reducing or eliminating the pathogen culture populations in the larger airways, likelihood of re-infection of the smaller airways and lung tissue is greatly reduced.

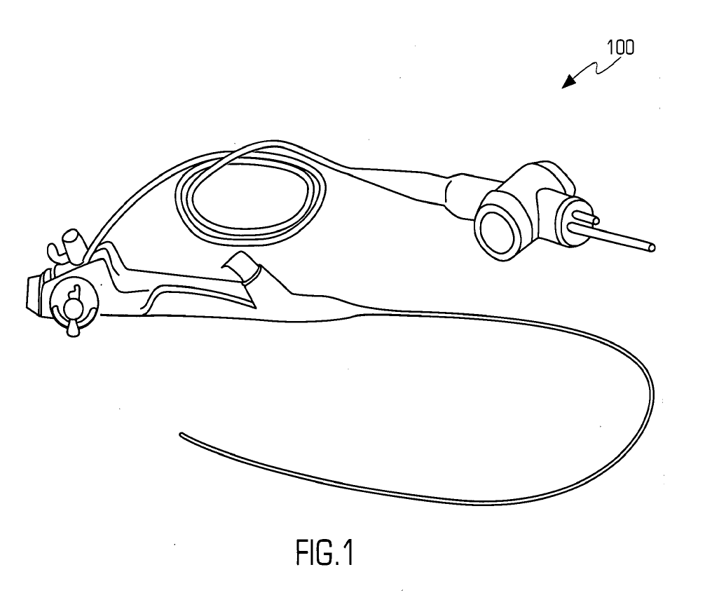

FIG. 1 shows a typical video bronchoscope 100 that can be modified or accessorized with the disclosed apparatus.

The disclosure is directed to methods and apparatus for the reduction and/or elimination of pathogens causing infection in human and animal respiratory systems and other body cavities. The method and apparatus can be used to treat infections occurring in patients having, for example, cystic fibrosis. The disclosure is also applicable to the disinfection of difficult to reach and access areas of inanimate objects as well.

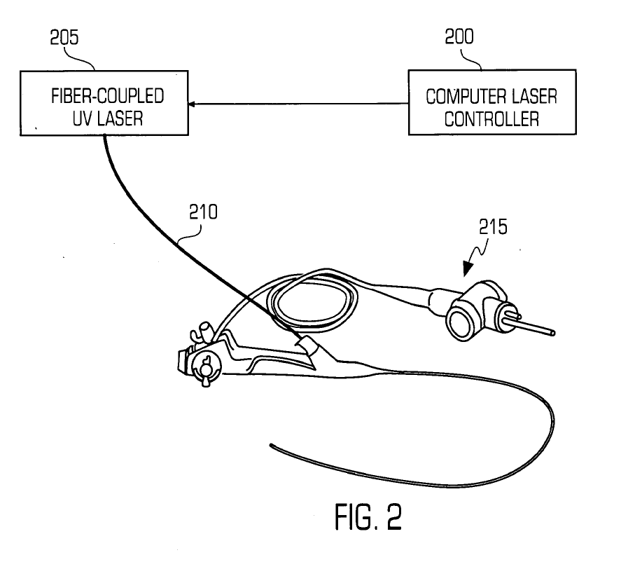

Continuing with a description of an application for lung therapy, FIG. 2 illustrates the block diagram of the apparatus of the disclosure. The video bronchoscope 215 is navigated by watching a monitor, attached in a well known manner and viewable by the medical professional operating the protocol, to visually guide the instrument to the desired area of the bronchial tree. This instrument is capable of reaching the distal end of each of the 18 segmental bronchi in the third generation of the bronchial tree in pediatric and adult patients. The computer laser controller 200 is used to set the duration, wavelength(s) and intensity of ultraviolet light to be applied. The wavelengths, duration and intensity of light to be used are predetermined based upon pathogen type(s) being killed, quantity and quality of mucous in infected airway area, size of patient, length of time allocated to overall procedure to be conducted and other factors. Other factors include the type of tissue being treated and its susceptibility to light induced damage and whether a “kill” or “cidal,” or a DNA deactivation or “-static” is desired. In some cases just deactivating DNA would be very valuable. The methods of treatment include protocols for the laboratory identification of pathogen(s) present and how they respond to different wavelengths of ultraviolet to determine optimal kill ratio with minimal risk of damage to respiratory system structures and tissue. The computer controller is connected to an appropriate fiber optic coupled light source or laser 205 functioning as a light source. Such fiber optic coupled lasers operating in the desired range are now available commercially. The light source has one or more computer controllable wavelengths, intensities and a shutter that can open and close to control duration of ultraviolet exposure. The light source 205 is in turn connected to a fiber optic cable 210 that is inserted into the open channel of the video bronchoscope 215 or is modified to utilize the visible light fiber optic system of the video bronchoscope that illuminates the viewing area for capture by the camera (often a charge coupled device camera) at the distal end of the video bronchoscope. The distal end of the fiber optic cable has a specially designed diffuser that illuminates a hemispherical area with approximately even distribution of light energy on all areas illuminated. The disclosure provides for treatment protocols including autoclaving and other sterilization procedures, for example UV sterilization, necessary to insure that infections are not spread from one patient to another. As mentioned above, FIG. 2 illustrates for lung therapy applications a device for computer controlled ultraviolet germicidal irradiation UVGI light source for fiber optically delivering the light to desired areas via an accessory for or modification to existing video bronchoscopes. The computer can control the duration, intensity and wavelength(s) of light being delivered during treatments. The disclosure includes methods for treatments of infected areas in a systematic manner that assures maximum pathogen kill ratios with minimal risk of tissue damage. The disclosed method and apparatus can work in conjunction with antibiotic drug therapies. One example is lung applications wherein the drugs perform the primary function of disinfecting small airways and tissue that are vascular and accessible via the circulatory system. In lung applications the disclosure provides the methods and apparatus to disinfect larger airways where greater mucous quantities are produced that creates an opportune environment for pathogen colonization and where the circulatory system does a poor job of delivery of intravenous or orally administered antibiotics. By reducing or eliminating the pathogen culture populations in the larger airways, likelihood of re-infection of the smaller airways and lung tissue is greatly reduced.

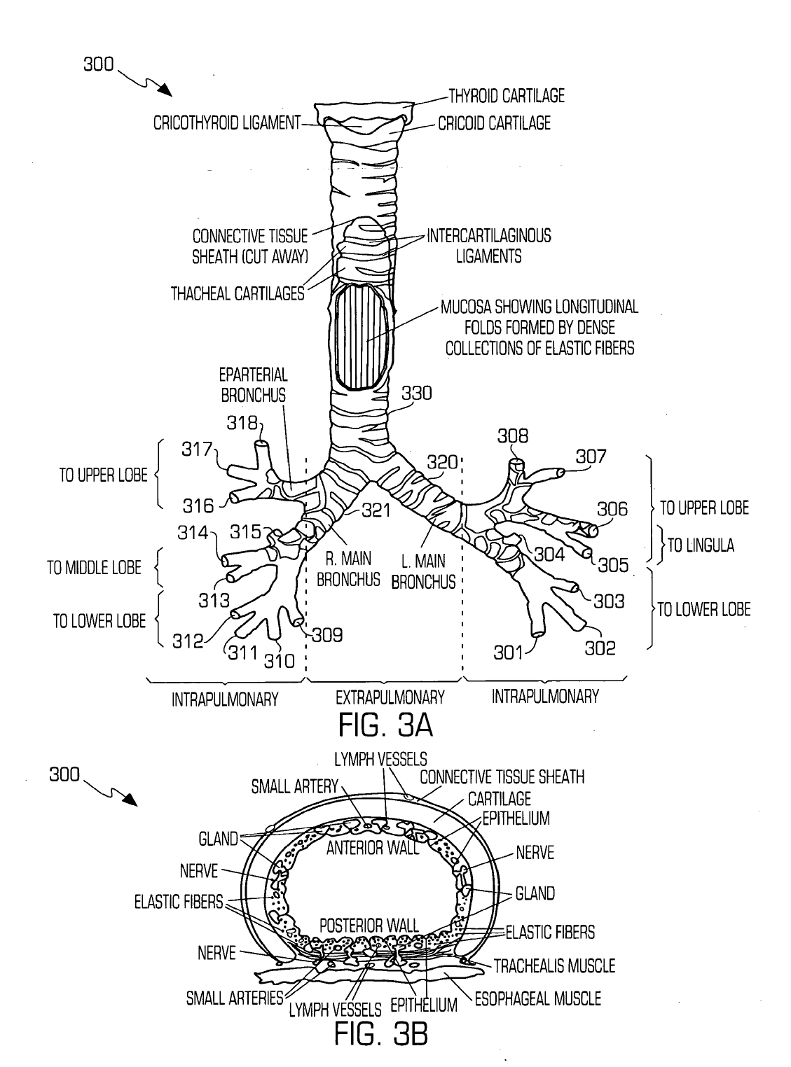

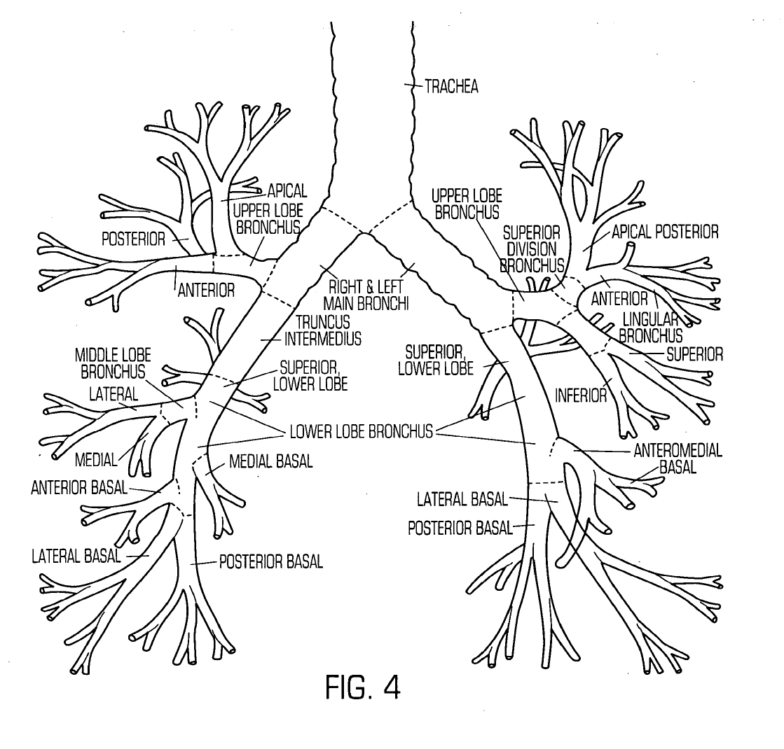

The disclosed method and apparatus provides treatment protocols including systematic process of delivery of uniform exposure of UVGI needed as predetermined during laboratory analysis of pathogen(s) cultured. FIG. 3 depicts the disclosure in a lung therapy application. As seen in FIG. 3, the proximal three generations of airways in the human respiratory system bronchial tree terminating in the 18 segmental bronchi. The basic treatment protocol begins by-instrument insertion into the distal end of the lower most segmental bronchi of the left lung 301 of the appropriately monitored and anesthetized patient. Once the distal end of this branch of the bronchial tree is in view on the monitor of the video bronchoscope, the predetermined settings for the UVGI light source are used to begin the exposure process. Next the physician or other appropriate medical professional performing the procedure withdraws the instrument at a predetermined rate as visually tracked on the monitor of the video bronchoscope until the intersection of the left main bronchus 320 is observed by seeing the proximal opening of the next lower most segmental bronchi of the left lung 302. The procedure is again performed for each subsequent next higher branch of the segmental bronchi in each lobe. Once the uppermost segmental bronchi branch of each lobe is treated (303 in the case of the left lower lobe), the main bronchus is then treated similarly perhaps using a different set of parameters of wavelength(s), duration and intensities to accommodate changes in cultures, airway size, or other known attributes, until the proximal opening of the lowest segmental bronchi branch (304 in the case of moving to the upper lobe of the left lung) of the next higher lobe becomes visible. At this point the procedure methodically begins over for each subsequent lobe, working from the bottom of the left lower lobe through the top of the left upper lobe 308 and then through the left main bronchus 320 to the junction of the trachea 330. Next the procedure continues starting with the lower most segmental bronchi of the lower lobe of the right lung 309 through the upper most segmental bronchi of the upper lobe of the right lung 318. Next the right main bronchus 321 is treated until the confluence of the trachea 330. Finally, the trachea is treated with appropriate predetermined settings applicable for known parameters of any particular patient's respiratory infection. While this procedure has been described the protocol beginning with the lower left segmental bronchi because it is the most distal, it will be appreciated by one of ordinary skill in the art that the protocol can begin with the right main bronchus. Also, it could be for specific airway regions of any of the five lobes only, and could also treat smaller airways down to the sixth generation airway as labeled in FIG. 4 to the fifth generation.

Use of perflourocarbons can provide additional applications for this patent. Perflourocarbons are used for “liquid ventilation” (LV) or “partial liquid ventilation” (PLV) of the lungs. These are fluids that can be taken into the lungs and the lungs can actually breathe the fluid. This gives rise to three additional applications for the present patent.

The first is an adaptation of the Video Bronchoscopic Germicidal Irradiation (“VBGI”) described above with respect to the device of FIG. 2. The liquid ventilation solution could be used directly or doped with an appropriate, additive such that UV light introduced through it by the device of FIG. 2 would reflect and refract into areas not accessible by the VBGI alone.

That is, the utilization of appropriately doped perflourocarbons or other so-called liquid ventilation (LV) or partial liquid ventilation (PLV) fluids in the lungs of humans and animals to reflect and refract UVC light will provide access to more surface area of the affected lung tissue being treated. With the lungs inflated with doped PLV (DPLV), the weight and pressure exerted on the lung tissue from the inside of the airway causes opening of airways and increases accessibility to otherwise inaccessible airways. Additionally, UV light being administered via the previously disclosed VBGI, can be more effective using DPLV that provides a liquid pathway for UV light to eradicate pathogens deeper in the lung bronchial tree illustrated in FIG. 4 than accessible by the previously disclosed bronchoscopic method alone.

The actual introduction of the liquid ventilation solution into the lungs or other appropriate body part can be done by today's well-known methods. For lung treatment, these methods include filling the lungs with the fluid. As the patient breathes, the fluid is used up and can be “topped off” continually or from time to time either manually or by use of a float valve. The introduction of the UV would be by VBGI perhaps requiring a different lens at the end of the bronchoscope device of FIG. 2 than would be used without the use of the solution. This may be a remotely controllable variable lens for different parts of the path in the lungs to control where the UV is being directed. Visible light can be used as a guide for this process. For example, depending on the refractivity of the liquid one may need to have a wide-angle lens to diffuse and disperse the UV light rather than focus the UV light.

Secondly, one can use the liquid ventilation solution with antibiotics to kill pathogens. Since one of the main reasons for the earlier disclosed apparatus and method is that aerosolized antibiotics generally do not reach the lungs effectively, this liquid ventilation delivery approach can improve the effectiveness of antibiotics. That is, by adding antibiotics that would normally be aerosolized and administered via breathing treatments to PLV, the antibiotics can be far more effective. These aerosolized antibiotics are usually inhibited from effectively functioning due to limited accessibility to pathogen-infected areas of the respiratory system. However, adding antibiotics to the above liquid ventilation delivery approach would improve their effectiveness.

The third application provides access to all or nearly all parts of the lung for retrovirus inoculation of gene transplant therapy. At present, advances in cystic fibrosis lung gene therapy are difficult due to lack of a delivery mechanism that is capable of reaching enough of the lung surface area to make a meaningful difference. By adding the “corrected gene” DNA carrying retrovirus to PLV fluids and then ventilating the patient using the fluid as disclosed above, the gene therapy would be able to treat a significant portion of the respiratory system surface area. It is commonly thought that greater than 10% of the respiratory surface area must be treated to achieve a meaningful change in respiratory function using gene therapy. By modifying the gene therapy procedure to use PLV, both greater effectiveness can be achieved and less frequent treatments are required.

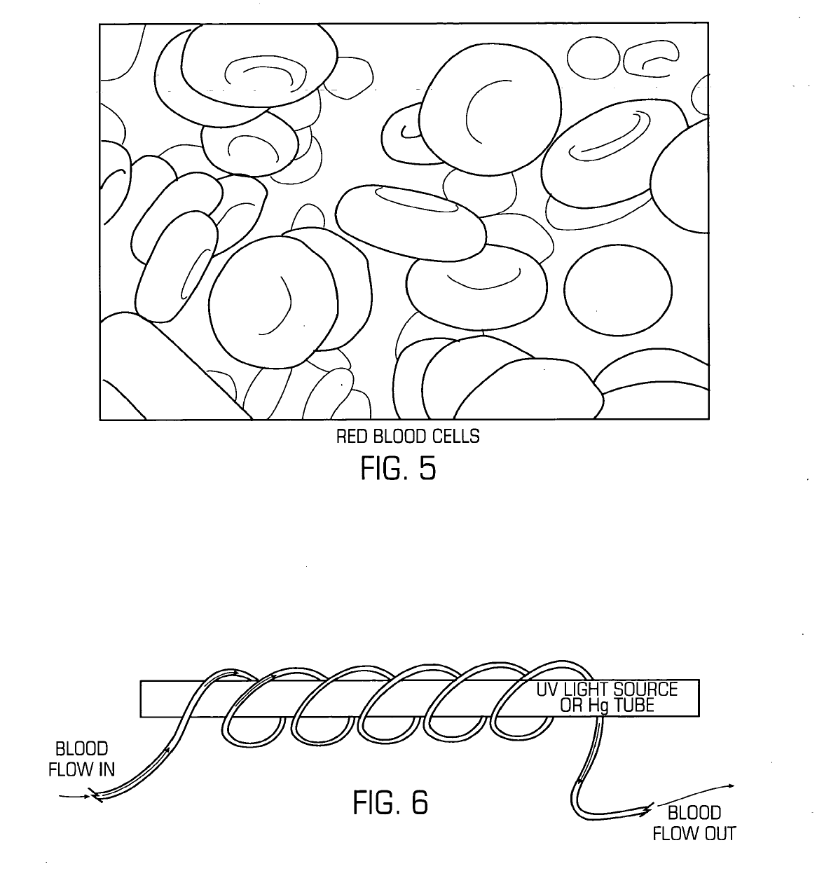

Another application of the disclosure can be for treatment of blood diseases. Referring to FIG. 5, there is illustrated a number of red blood cells and their donut shape. It is well know that most pathogens (viral, bacterial, fungal and chemical, as examples) adhere to the outside of the donut shape of the cell at least initially. It is also well known that most of these pathogens can be eradicated or deactivated by the application of UV light in the wavelength range of approximately 200 nm to 320 nm. The teachings of the disclosure can be applied to treating blood cells via a device similar to that illustrated in FIG. 6. In that device a UV light source, which could be a bulb or a tube such as a mercury tube, is wrapped with a quartz coil that exposes blood cells passing though it to UV light.

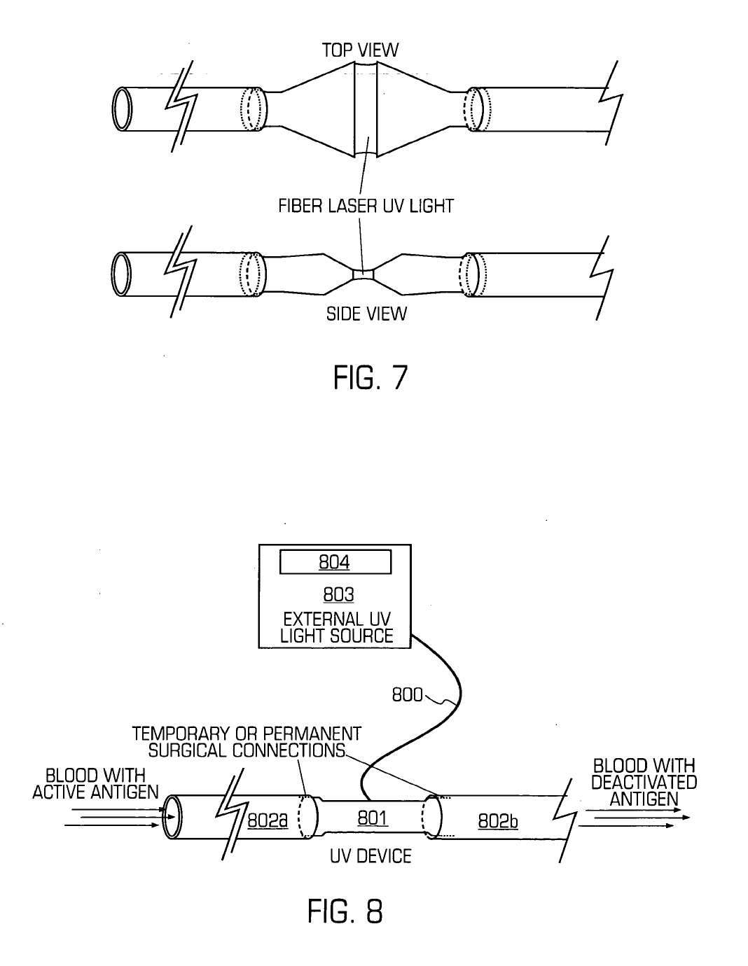

FIG. 7 illustrates another embodiment useful in treating blood diseases. In the device illustrated in that figure, a tube through which blood flows is connected to a flanged or other suitably shaped area where it flattens out and quartz or other suitable material window is fitted with a fiber optic UV light source such as the fiber coupled laser discussed above. The devices of FIG. 6 or FIG. 6 can be shrouded to prevent UV exposure outside the desired exposure areas. The coil in FIG. 6 and the flattened bridge device in FIG. 7 can be disposable, or can be autoclavable for subsequent use. Either device can be fitted inline to heart-lung machines or other suitable apparatus for blood treatment of a patient external to the patient's body. Since the DNA of blood cells is not used for replication or reproduction inasmuch as blood is made in the marrow of bones, the UV light that damages DNA will deactivate the pathogen DNA with little or no harmful effect on the blood cell's functionality. The UV irradiated blood can then be passed back into the patient's body where the deactivated pathogens are not able to replicate, and they can eventually be removed via the patient's immune system.

FIG. 8 illustrates an additional embodiment for bodily fluid treatment. This is a small, permanent or temporary, surgically installed, inline arterial (or other bodily tube for bodily fluid other than blood) germicidal irradiation blood or other bodily fluid treatment device 801. It could have its UV light source external to the body, which would be connected via a fiber optic coupling 800 as needed during periodic treatment. Treatment could be in-home, in hospital or as an outpatient in a doctor's office or other suitable office or center. This could be used for treatment against HIV/AIDS, leukemia and/or other blood borne (or other bodily fluid borne) pathogens.

As seen in FIG. 8, there could be a permanent or temporary surgical connection to UV device 801 between parts of an artery, vein or other bodily fluid conducting tube 802(a), 802(b). The device 801 can be constructed so as to have internal baffling (not shown) or other turbulence-inducing construction. The internal baffling can cause fluid flow through the device to become turbulent therefore exposing more surface area of the fluid passing through the device to UV as desired. The connection of the device 801 with artery or vein or other bodily fluid conducting tissue can be permanent or temporary and is surgically implanted in connecting relationship between two sections of the artery, vein or other tissue. The device is preferably constructed with inert plastic. It can be made such that connective tissue, such as artery, vein or other, as appropriate, is not exposed to UV. That is, the device itself acts to contain essentially all the UV light and exposes only the fluid passing through it to UV, as explained with respect to an earlier embodiment. The device can have a remote or external UV light source connected via fiber optic or other suitable coupling 800 for the period of the treatments depending upon the pathogen, patient health, an other criteria. The external light source 803 can be a fiber coupled UV laser, as described above, or other appropriate UV light source. Sometimes the connection of the external light source to the patient is called a button, which refers to the patient's connection point to the external light source. What is required is the connection to the external light source, here preferably a fiber optic connection, and a good mechanical connection surgically to the patient's tissue at the connection site to keep the fiber optic cable connected to the UV treatment chamber 801 within the patient from pulling out or entangling with other structures in the patient's anatomy. In operation, the fluid would pass through the device or treatment chamber 801 to allow UV light to irradiate the fluid flowing through the device at appropriate periods. Digital or analog control means, well known in the art, can be used to control the frequency, time period and intensity of the UV light as it is exposed to the fluid flowing through the device 801.

While the foregoing description has been with reference to particular embodiments, it will be appreciated that these are only illustrative and that changes may be made to those embodiments without departing from the principles of the invention, the scope of which is defined by the spirit and scope of this overall description.

What is claimed is:

1. An apparatus comprising:
a laser for providing irradiation in the ultraviolet spectrum;
a computer controller coupled to the laser;
an optical fiber coupled to the laser to transmit the ultraviolet irradiation; and
a tube attached to one end of the optical fiber, wherein the optical fiber delivers the ultraviolet irradiation into the tube so as to irradiate a patient's blood or bodily fluids flowing through the tube, the blood or bodily fluids including pathogens therein;
the computer controller setting at least one of the duration, wavelength(s) or intensity of ultraviolet irradiation to be applied by the laser to deactivate the DNA of the pathogens.

2. The apparatus of claim 1 wherein the tube is configured to be placed within the patient's body inline to an artery or other bodily fluidic passage.

3. The apparatus of claim 2 wherein the pathogens comprise one or more of bacteria, virus or fungus.

4. The apparatus of claim 1 wherein the tube is configured to be placed external to the patient's body with the blood or other bodily fluids diverted to flow through the tube.

5. The apparatus of claim 4 wherein the pathogens comprise one or more of bacteria, virus or fungus.

6. An apparatus comprising:
a laser for providing irradiation in the ultraviolet spectrum;
a computer controller coupled to the laser; and
an optical fiber coupled to the laser to transmit and deliver the ultraviolet irradiation to one or more toenails, the one or more toenails including Trichophyton Rubrum thereon; and
the computer controller causing the laser to deliver the ultraviolet irradiation with at least one of a duration, wavelength(s) or intensity sufficient to treat the Trichophyton Rubrum with minimal damage to surrounding tissue of said one or more toenails.

7. The apparatus of claim 6 wherein the minimal damage to surrounding tissue of said one or more toenails includes avoiding mutagenicity and carcinogenicity.

8. The apparatus of claim 6 wherein treating the Trichophyton Rubrum includes deactivating the DNA of the Trichophyton Rubrum.

9. An apparatus comprising:
a laser for providing irradiation in the ultraviolet spectrum;
a computer controller coupled to the laser;
an optical fiber coupled to the laser to transmit and deliver the ultraviolet irradiation to one or more areas of a patient's skin, said one or more areas of the patient's skin including Pseudomonas Aeruginosa thereon; and
the computer controller causing the laser to deliver the ultraviolet irradiation with at least one of a duration, wavelength(s) or intensity sufficient to treat the Pseudomonas Aeruginosa with minimal damage to surrounding tissue of said one or more areas of a patient's skin.

10. The apparatus of claim 9 wherein the minimal damage to surrounding tissue of said one or more areas of a patient's skin includes avoiding mutagenicity and carcinogenicity.

11. The apparatus of claim 9 wherein treating the Pseudomonas Aeruginosa includes deactivating the DNA of the Pseudomonas Aeruginosa.

12. The apparatus of claim 11 wherein the one or more areas of the patient's skin contain burn wounds infected by Pseudomonas Aeruginosa.

13. A method comprising:
diagnosing a type of a pathogen infecting bodily fluids in a human patient;
selecting UV irradiation parameters comprising at least one of the duration, wavelength(s) or intensity to deactivate the DNA of the pathogen but below a threshold for minimal damage to the bodily fluids; and
delivering the UV irradiation with the selected parameters through a fiber-coupled laser apparatus controlled by a computer regulated routine so as to deactivate the DNA of a pathogen in the tissue.

14. The method of claim 13 wherein the pathogen comprises one or more of bacteria, virus or fungus.

15. A method comprising:
diagnosing a type of a pathogen infecting a tissue in one or more areas on the exterior of a human body;
selecting UV irradiation parameters comprising at least one of the duration, wavelength(s) or intensity to deactivate the DNA of the pathogen but below a threshold for minimal damage to the tissue; and
delivering the UV irradiation with the selected parameters through a fiber-coupled laser apparatus controlled by a computer regulated routine so as to deactivate the DNA of a pathogen in the tissue.

16. The method of claim 15 wherein the minimal damage to the tissue includes avoiding mutagenicity and carcinogenicity.

17. The method of claim 15 wherein the pathogen comprises one or more of bacteria, virus or fungus.

18. The method of claim 15 wherein the pathogen is Trichophyton Rubrum.

19. The method of claim 15 wherein the pathogen is Pseudomonas Aeruginosa.

20. A method comprising:
diagnosing the type of a pathogen infecting a tissue in the lung of a human or animal;
opening the airways inside the lung by using doped perfluorocarbons in a liquid ventilation or partial liquid ventilation process in the lung;
selecting UV irradiation parameters comprising at least one of a duration, wavelength(s) or intensity to deactivate the DNA of the pathogen but below a threshold for minimal damage to the tissue;
delivering UV irradiation with the selected parameters transmitted by a fiber-coupled laser apparatus so as to deactivate the DNA of a pathogen in the tissue; and
the doped perfluorocarbons selected for optical properties to refract or reflect the UV irradiation.

21. The method of claim 20 wherein the fiber-coupled laser apparatus is controlled by a computer regulated routine.

22. The method of claim 20 wherein the liquid ventilation or partial liquid ventilation process further comprises antibiotics with the doped perfluorocarbons.

23. The method of claim 20 wherein the liquid ventilation or partial liquid ventilation process further comprises retrovirus carrying DNA for gene therapy with the doped perfluorocarbons.

24. The method of claim 20 wherein the minimal damage to the tissue includes avoiding mutagenicity and carcinogenicity.
